(19) United States
(12) Patent Application Publication
(10) Pub. No.: US 2022/0339397 A1
(43) Pub. Date: Oct. 27, 2022

(54) SENSORY STIMULATION BOOTH

(71) Applicant: Louis Michael Bertram, Vanceburg, KY (US)

(72) Inventor: Louis Michael Bertram, Vanceburg, KY (US)

(21) Appl. No.: 17/730,888

(22) Filed: Apr. 27, 2022

Related U.S. Application Data
(60) Provisional application No. 63/180,307, filed on Apr. 27, 2021.

(51) Int. Cl. A61M 21/00; A61M 21/02; H04R 5/02

(52) U.S. Cl. CPC A61M 21/0094; A61M 21/02; H04R 5/023; A47C 7/72

(57) ABSTRACT

A sensory stimulation booth. The booth includes an enclosure having a floor, a ceiling, and a plurality of walls positioned around the periphery of the floor and the ceiling to collectively define an interior space therebetween. The booth further includes a plurality of speakers and a plurality of light fixtures within the enclosed space that are all oriented towards a common focal point. Also disclosed are embodiments of the booth featuring tactile transducers, vaulted ceilings, and isolation feet.

### CROSS-REFERENCE TO RELATED APPLICATIONS

This application is a non-provisional patent application that makes a priority claim to U.S. Provisional Application No. 63/180,307, which is incorporated by reference as if fully recited herein.

### TECHNICAL FIELD

Exemplary embodiments relate generally to a booth and method for stimulating the auditory and visual senses of a user.

### BACKGROUND

Ayurveda and Traditional Chinese medicine (commonly abbreviated “TCM”) are systems of holistic healing, health, and wellness that are native to India and China, respectively. Each of these systems have been developed over the course of thousands of years and are now practiced worldwide (including the United States).

One principal that is common to both Ayurveda and TCM is the notion that the human body comprises a plurality of energy systems. In Ayurveda this is known as the seven chakras that align across the length of the spine; and in TCM this is known as the qi that flows though the Meridian System (i.e., a network of qi pathways within the human body). These energy systems are believed to be in constant communication with one another and subject to change. When one or more of these energy systems is blocked or otherwise imbalanced, it is believed that this deviation from an otherwise balanced state can be an attributing factor to illness or disease. And, by this principal, it may be possible to achieve a sense of wellness by bringing these energy systems back into a state of balance.

Accordingly, it is contemplated that apparatus, systems, and methods for rebalancing the energy systems of a person would be desired.

### SUMMARY OF THE INVENTION

Disclosed are sensory stimulation booths that include an enclosure and a plurality of speakers and a plurality of light fixtures within the enclosure.

In one embodiment, the sensory stimulation booth includes an enclosure. The enclosure includes a floor, a ceiling, and a plurality of walls positioned around the periphery of the floor and the ceiling to collectively define an interior space therebetween. The booth further includes a plurality of speakers and a plurality of light fixtures within the interior space. Each speaker and each light fixture is oriented towards a focal point within the interior space.

In another embodiment, the sensory stimulation booth includes an enclosure. The enclosure floor, a ceiling, and a plurality of walls positioned around the periphery of the floor and ceiling in a polygonal arrangement. The floor, ceiling, and walls define an interior space therebetween. The booth further includes a plurality of speakers and a plurality of light fixtures within the interior space. Each speaker and light fixture is oriented towards a focal point within the interior space.

In yet another embodiment, the sensory stimulation booth includes an enclosure. The enclosure includes a floor, a vaulted ceiling, and a plurality of walls positioned around the periphery of the floor and the vaulted ceiling in a hexagonal arrangement. The floor, vaulted ceiling, and walls defining an interior space therebetween. The sensory stimulation booth further includes a plurality of isolation feet underneath the floor of the enclosure. The sensory stimulation booth further includes a plurality of speakers and a plurality of light fixtures within the interior space. Each speaker and each light fixture is oriented towards a single point within the interior space.

Other examples of the disclosed sensory stimulation booth, and methods of using, with become apparent from the following detailed description, the accompanying drawings, and the appended claims.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1 is a front elevational view of a first embodiment of a sensory stimulation booth;

FIG. 2 is a top cross-sectional view of the booth of FIG. 1;

FIG. 3 is a side cross-sectional view of the booth of FIG. 1;

FIG. 4 is an elevational view of an enclosure wall with a frame;

FIG. 5 is an elevational view of an enclosure wall with a frame;

FIG. 6 is an elevational view of an enclosure wall with a door;

FIG. 7 is a front elevational view of a second embodiment of a sensory stimulation booth;

FIG. 8 is a side cross-sectional view of the booth of FIG. 7;

FIG. 9 is a top view of the booth of FIG. 7;

FIG. 10 is a bottom view of the booth of FIG. 7;

FIG. 11 is a front elevation view of an isolation foot of the booth of FIG. 7;

FIG. 12 is a table diagram depicting the association between the Seven Chakras of Ayurveda with certain human organs, musical tones, and colors of light; and

FIG. 13 is an illustration showing the general locations of the Seven Chakras of Ayurveda within the human body.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

Embodiments of the invention are described herein with reference to illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

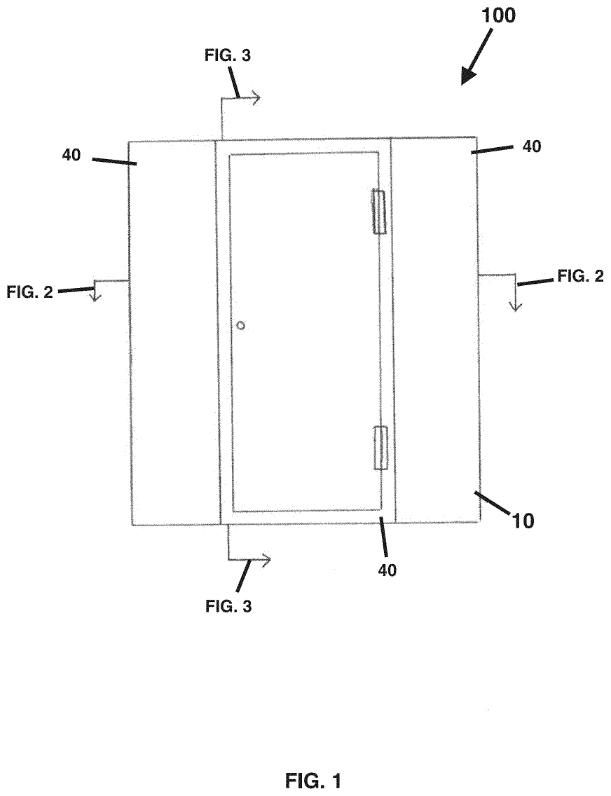

Referring to FIGS. 1-3, depicted is an exemplary embodiment of a sensory stimulation booth 100 (herein, the “booth”). The booth 100 is designed to provide an optimal environment for stimulating the auditory and/or visual senses of a user. The booth 100 may be used for therapeutic purposes or simply for enjoyment. As shown, the booth 100 includes an enclosure 10 having a floor 20, a ceiling 30, and a plurality of walls 40; with the floor 20, ceiling 30, and walls 40 defining an interior space 50 therebetween. The booth 100 also includes a plurality of speakers 60 and a plurality of light fixtures 70 within the interior space 50. By stepping into the booth 100 and activating the speakers 60 and light fixtures 70, a user can stimulate his/her auditory and visual senses by being immersed in sound and light.

In the embodiment shown, enclosure 10 features a six-walled hexagonal design. Its overall shape is that of a hexagonal prism, with the six walls 40 each being congruent in size and shape. It is contemplated that in this embodiment, as well as in other embodiments, the floor 20 and the ceiling 30 may be shaped in accordance with the walls 40 to ensure that an isolated, interior space 50 is achieved (i.e., substantially devoid of any cracks, openings, etc., except as needed to provide for ventilation). Here, for example, the floor 20 and the ceiling 30 are shaped as regular hexagons (which is the cross-sectional shape of a hexagonal prism).

While enclosure 10 is considered an exemplary embodiment, it is not meant to be limiting. Other embodiments of the enclosure may feature different polyhedral designs, or alternatively, cylindrical (including elliptic cylinders) and even irregular designs. These differences are not considered a departure from the scope of the present disclosure. Further, like the embodiment of FIGS. 1-3, the floors and ceilings of those enclosures may be shaped as appropriate to match the shape of the walls (i.e., the cross-sectional shape when viewed from above).

The structural components of the enclosure 10 (i.e., the floor 20, ceiling 30, and walls 40) may each be fabricated using any suitable material such as, but not limited to, wood, stone, and/or metal. In general, however, it is contemplated that wood may be preferred due to its relatively low cost, light weight, and commercial availability. For example, it is contemplated that the walls 40 may be fabricated from prefinished tongue and groove poplar boards; and that the floor 20 and ceiling 30 may be fabricated from ¾″ particle board.

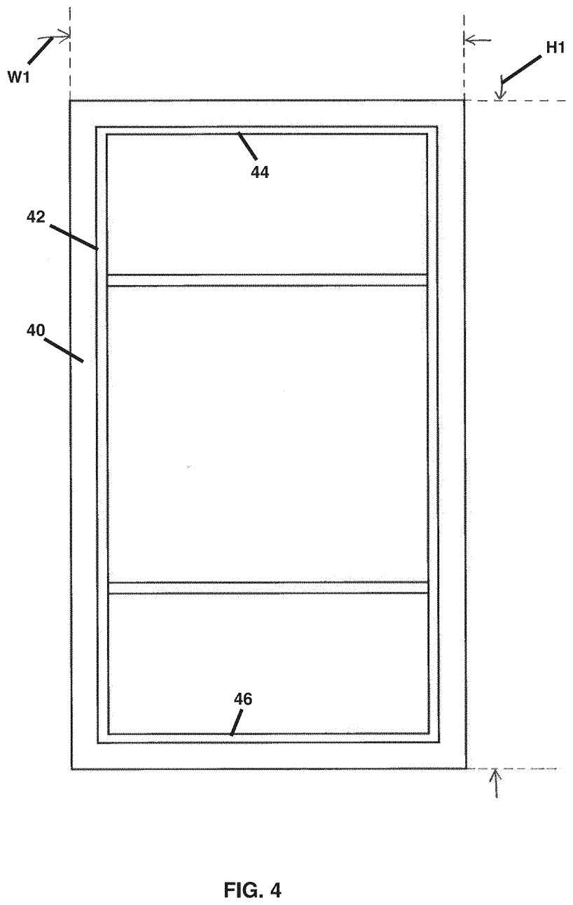

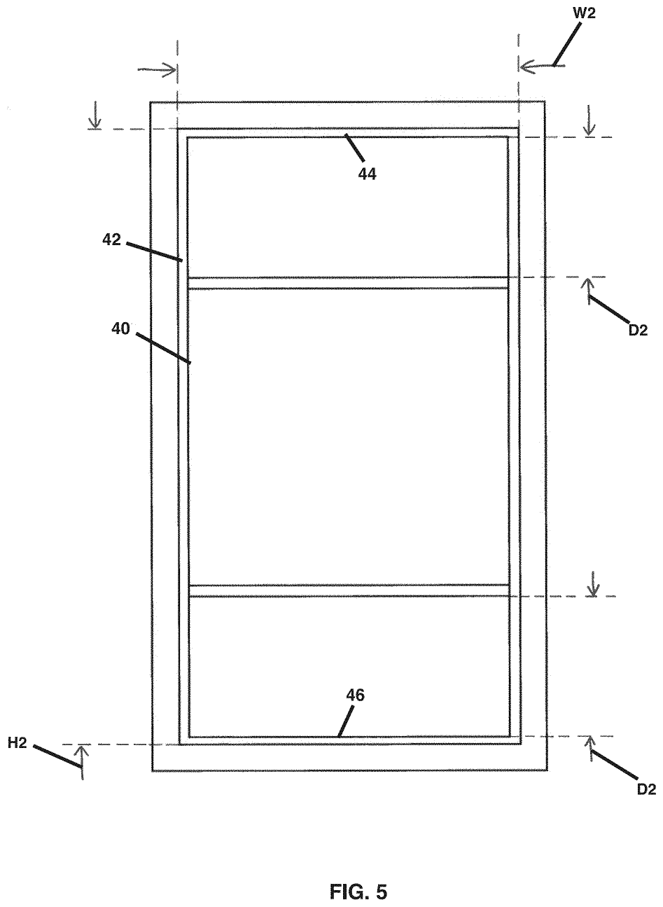

Referring to FIGS. 4-5, a frame 42 may be provided to reinforce a wall 40. The frame 42 may be, for example, a ladder frame (as shown) that encompasses most of the interior surface of a wall 40. These frames 42 may be fabricated from, for example, 2-inch-by-2-inch wooden boards. These frames 42 may be affixed to the walls 40 by any suitable means such as, for example, adhesives or mechanical fasteners.

In practice, the frames 42 may also help facilitate the attachment of the walls 40 to the floor 20 and/or the ceiling 30. In the embodiment shown, the frame 42 can serve as a bracket by utilizing, for example, top horizontal member 44 and a bottom horizontal member 46 to secure the frame 42 to the ceiling 30 and the floor 20, respectively. These can be secured by way of, for example, mechanical fasteners such as galvanized carriage bolts and the like.

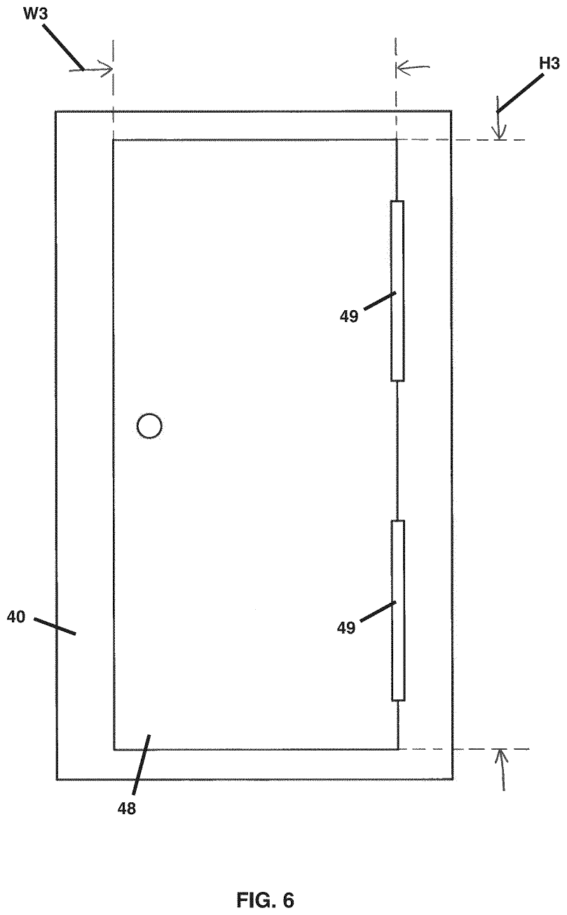

Referring to FIG. 6, a door 48 may be provided on one or more of the walls 40 to permit entry and exit out of the interior space 50. The door 48 may be secured by way of hinges 49. Any suitable type of door 48 and hinge 49 may be utilized here such as, for example, a door fabricated from tongue and groove poplar boards that is secured by 30-inch piano hinges.

The overall size of the enclosure 10 may be varied without departing from the scope of the present disclosure. In general, it is contemplated that the enclosure 10 should be large enough for at least one adult user to step through the door 48 and comfortably remain therein. Preferably, the user may also comfortably sit therein. In other embodiments, it is also completed that the enclosure 10 may be scaled up in size so as to accommodate a larger number of users.

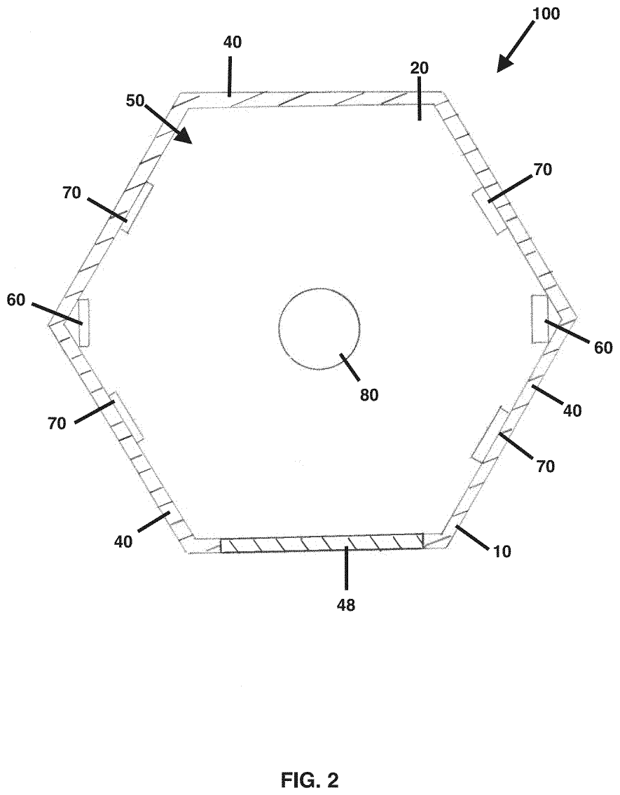

The present disclosure will now provide a specific embodiment of an enclosure that is suitable for use on an individual basis (i.e., one user at a time). In this embodiment, each of the six walls may be 36 inches wide (W₁) and 86 inches in height (H₁) (FIG. 4). These walls may be reinforced with a frame that is 34 inches wide (W₂) and 81 inches in height (H₂) (FIG. 5). This frame may feature a top horizontal member and a bottom horizontal member each attached to opposing axial ends of two vertical members. This frame may also include two additional horizontal members disposed 21 inches inward relative to the top and bottom horizonal members (D₂). Further, this booth may include a door on one of the walls that is approximately 26 inches wide (W₃) and 77 inches in height (H₃) (FIG. 6). This door may be secured to the wall with two 30-inch piano hinges. One piano hinge may be attached to the wall approximately 6 inches from the top and the other 6 inches from the bottom. The base and the ceiling may be regular hexagons in shape, having a center-to-vertex dimension of approximately 36 inches (FIG. 2).

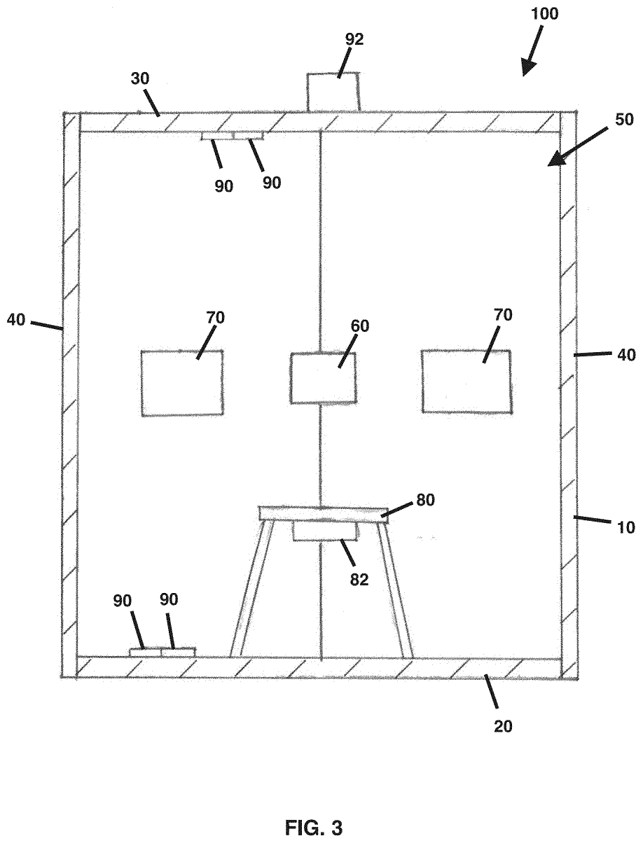

Referring back to FIGS. 2 and 3, light fixtures 70 may be provided within the interior space 50. The booth 100 of the embodiment shown includes four light fixtures 70—one positioned along each of the front right, front left, rear right, and rear left walls 40. These light fixtures 70 may be mounted directly to the walls 40 (e.g., 18 inches from the floor) and oriented towards a focal point within the interior space 50 (e.g., the center of the enclosure where seat 80 is located). By this arrangement, a user who steps into the interior space 50 and positions him/herself at the focal point may receive direct light from substantially all directions, thereby maximizing light immersion. Any suitable light fixtures 70 may be utilized in the booth. However, it is contemplated that light fixtures 70 capable of generating a range of colors are preferred (e.g., RGB and RGBW LED lights). As those skilled in the art will appreciate, “RGB” stands for red-green-blue; “RGBW” stands for red-green-blue-white; and “LED” stands for light-emitting diode. Additionally, it is also contemplated that light fixtures 70 having a brightness ranging from about 8,000 lux to about 12,000 lux is preferred, or even more preferably about 10,000 lux. One specific example of a light fixture 70 that may be suitable for such use can include the COLORrail IRC, which is an LED light fixture available from Chauvet & Sons, LLC of Sunrise, Fla.

Of course, other embodiments of the booth may vary in the quantity, type, and arrangement of light fixtures 70 without departing from the scope of the present disclosure. For example, it is contemplated that the light fixtures 70 may be attached or positioned on the floor 20 or the ceiling 30. In another example, it is contemplated that either more or less light fixtures 70 may be included (e.g., on the front wall and/or on the rear wall).

Speakers 60 may be provided within the interior space 50. In the embodiment shown, the booth 100 includes two speakers 60—one positioned within each of the furthest left and furthest right corners. Any suitable type of speaker 60 may be utilized, but it is contemplated that speakers 60 having a sensitivity rating of less than 100 decibels is preferred, or even more preferably less than 85 decibels. It is further contemplated that speakers having a frequency range of about 1 hertz to about 20,000 hertz is preferred. A specific example of a speaker 60 that may be suitable here can include 150-watt powered bookshelf stereo speakers available from Audioengine, LLC. of Austin, Tex.

The arrangement of speakers 60 within the booth 100 may vary without departing from the scope of the present disclosure. To maximize the immersive aspect, it is contemplated that the speakers should ideally surround the user from multiple (if not all) directions. Further, the speakers 60 would ideally be oriented towards a focal point within the interior space 50, ideally the same focal point for the light fixtures 70. Additionally, it is contemplated that orienting the speakers 60 towards a corner in the enclosure 100 would be preferred since the soundwaves emitted by that speaker 60 would disperse upon hitting the corner, which reduces, if not eliminates, any 1^{st} order reflections. Owing to this effect, it is contemplated that hexagonal enclosures may be preferred since the hexagonal shape ensures that there is always a corner opposite a speaker 60 no matter which corner the speaker 60 is placed in.

To improve sound quality within the interior space 50, it is further contemplated that the booth 100 may include one or more acoustic features for absorbing and/or diffusing sound. These acoustic features can include, for example, baffles, sound-absorbing panels, foam, carpeting, and the like. Further, these acoustic features can be positioned along the floor 20, ceiling 30, and/or walls 40; or wherever is appropriate to achieve the highest quality sound within the interior space 50. In one embodiment, for example, the booth may include carpeting on the floor 20 and ceiling 30.

In addition to auditory and visual stimulation, the booth may be configured for tactile stimulation as well. Referring to FIG. 2, it is contemplated that the booth may include one or more seats 80 (e.g., stools, chairs, etc.), with each seat incorporating one or more tactile transducers 82 (aka, “bass shakers”). A user can sit on a seat 80 and receive tactile vibrations from a tactile transducer 82 to stimulate his/her vibrotactile senses. More than one seat 80 with tactile transducer(s) 82 may be included within the booth 100 (e.g., one for each user). These tactile transducers 82 can be activated either independently of, or in coordination with, the lights and speakers.

The embodiment shown in FIG. 3 features a stool-type seat 80 with a single tractile transducer 82 installed underneath. The tactile transducer 82 is oriented upwards so that tactile vibrations may be directed towards a user sitting on the stool. Preferably, the tactile transducer 82 would be able to produce vibrations having a frequency range of 5 hertz to about 200 hertz, or more preferably about 20 hertz. A specific example of a tactile transducer 82 that may be suitable for this application may be the AST-2B-4, which is a tactile 50-watt bass frequency shaker available from AuraSound, Inc. of Santa Fe Springs, Calif.

In embodiments where seat 80 is a chair having a back rest, it is contemplated that tactile transducers can be provided on the backrest either in addition to, or as an alternative for, a tactile transducer positioned underneath the seat. In a preferred embodiment, a plurality of tactile transducers may be aligned in a row and positioned along the backrest of the chair where the spine of a user would be.

It is further contemplated that tactile transducers may be incorporated into the booth without a seat; and that a seat may be incorporated without tactile transducer(s). For example, it is contemplated that tactile transducers may be incorporated into the floor 20 or the walls 40 of the enclosure 10. Configurations such as these may be more appropriate for applications that involve having the user stand within the booth 100, rather than sit.

Power can be supplied to the speakers 60, light fixtures 70, and/or tactile transducers 82 though one or more electrical outlets 90 (e.g., 15-amp, 120 volt, three-pronged wall outlets) provided on the enclosure 10. Referring to FIG. 3, for example, the booth 100 includes four electrical outlets 90—two mounted on the ceiling 30 facing down, and two mounted in the floor 10 facing up (wiring not shown). These electrical outlets 90 may be routed to a junction box 92 (shown as being mounted on top of the enclosure 10) that is supplied power through a wired connection to an external power source. The wired connected may include, for example, 12-2 wire (i.e., a 12-gauge wire comprising 2 insulated current carrying wires and a bare ground wire). Of course, the number and placement of electrical outlets 90 and junction boxes 92 need not be a limiting feature. For example, in other embodiments the booth 100 may include either more or less wall outlets 90 to accommodate either more or less light fixtures 70 and speakers 60. In yet other embodiments, the electrical outlets 90 and/or junction box 92 may be positioned elsewhere on the enclosure 10 (i.e., on the walls 40 or entirely on the floor 20). In still another embodiment, it is also contemplated that instead of providing a junction box 92 and electrical outlets 90, the speakers 60 and light fixtures 70 may be supplied power directly from an external power source.

The speakers 60, light fixtures 70, and tactile transducers 82 may be operatively connected to a computer, or similar electronic device (e.g., smartphone), to enable an operator to interface with and control the booth 100. It is contemplated that the operator may, but need not, be the same person as the user. Operative connectivity may be achieved by any suitable means including both wired and wireless solutions (e.g., WIFI, Bluetooth, etc.). One example off a software solution that may be suitable here can include ShowXpress available from Chauvet & Sons, LLC of Sunrise, Fla.

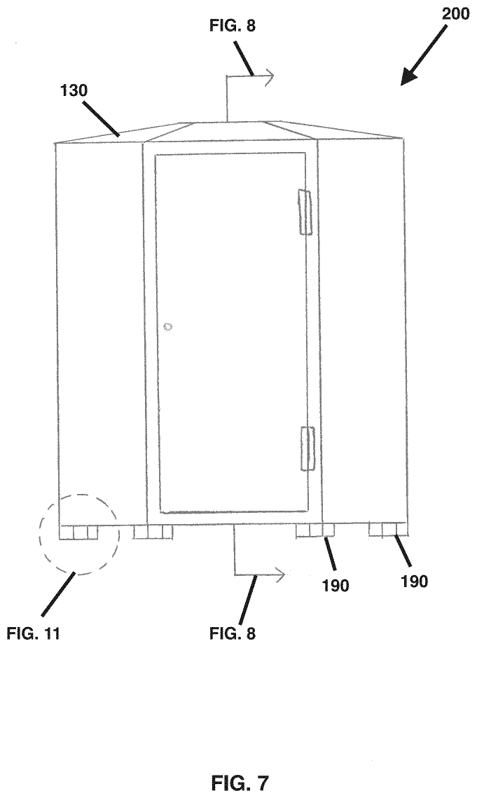

FIG. 7 shows an embodiment of the booth 200 having a vaulted ceiling 130 rather than the flat (i.e., horizontally planar) ceiling 30 shown in FIGS. 1-3. It is contemplated that a vaulted ceiling 130 is an alternative design that may be incorporated to improve sound quality and/or the immersive aspect of the booth 200. Without being bound by any particular theory, its believed that a vaulted ceiling 130 may help to redirect or focus sound waves from the speakers towards a focal point within the interior space (which is where user(s) are supposed to be positioned).

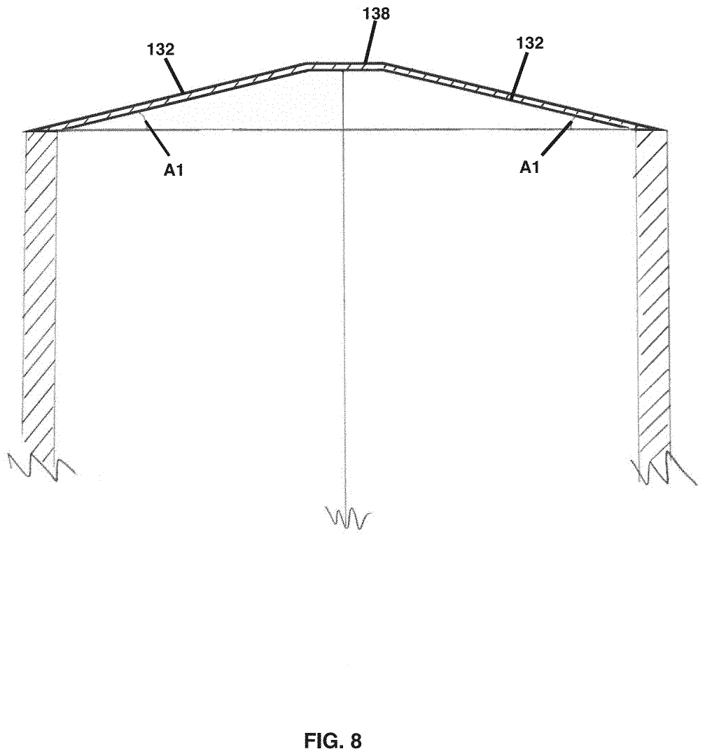

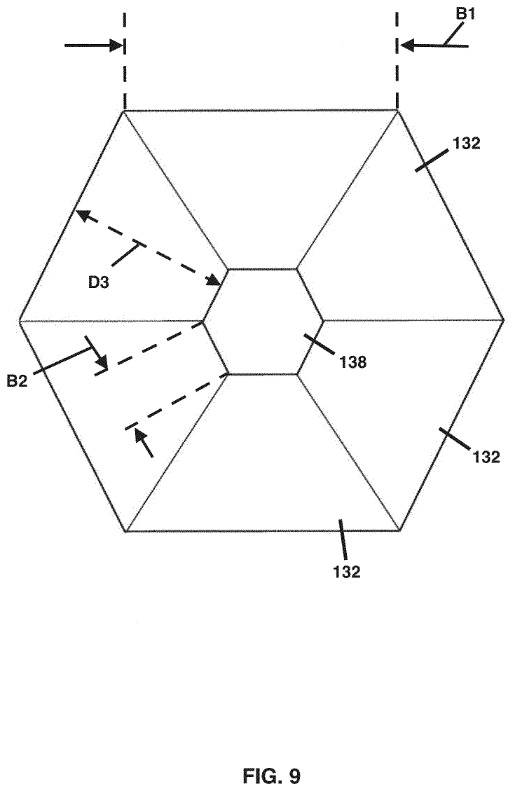

Referring to FIGS. 8-9, the vaulted ceiling 130 of FIG. 7 has the shape of a truncated hexagonal pyramid, with sloping sides 132 and flat top 138. Each of the sloping sides 132 are trapezoidal in shape, with the flat top 138 being a regular hexagon (FIG. 9). The major base 134 of the sloping sides may be, for example, 33.5 inches in width (B₁). The minor base 136 of the sloping sides may be, for example, ______ inches in width (B₂). The height of the sloping sides 132 may be, for example, 24 inches in height (D₃). The sloping sides 132 may have a slope angle A₁ ranging from 3-20°, or more preferably between 4-10°. In a preferred embodiment, the slope angle θ may be 4.76°.

It is contemplated that one or more light fixtures 170 may be attached to the inside surfaces of the sloping sides 136 and/or the flat top 138 to immerse a user in light from above. These light fixtures 170 may also be oriented towards a focal point within the interior space.

Of course, as those skilled in the art will appreciate, vaulted ceilings can be fabricated in various shapes (e.g., hemispherical domes) while still producing the same or similar effect (i.e., redirecting sound waves). These varying vaulted ceiling designs may also be incorporated without departing from the scope of the present disclosure.

Also shown in FIG. 7 are isolation feet 190. Isolation feet 190 can be installed on the booth as a way of blocking vibrations from the speakers and/or tactile transducers from being transmitted to the ground, thereby minimizing (if not eliminating) any possibility of vibrational resonance. Thus, it is contemplated that the isolation feet 190 can improve sound quality while also improving the overall stability of the booth 200.

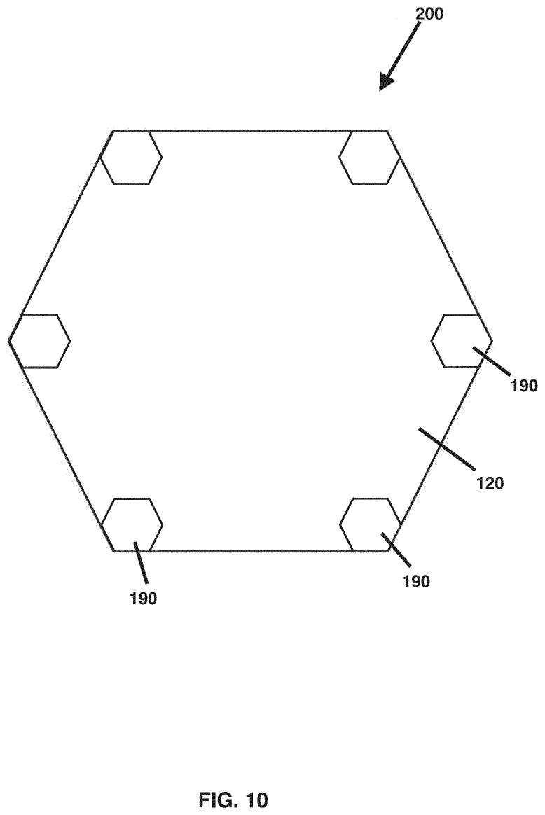

Referring to FIG. 10, the booth 200 features six isolation feet 190 positioned along the vertexes of the hexagonal floor 120. Each of these isolation feet 190 are also hexagonal in shape. Ideal dimensions for the isolation feet 190 may range from, for example, 5 inches to 10 inches in length (as measured between diametrically opposed vertexes), or more preferably between 7 inches and 8 inches in length, or even more preferably about 7.78 inches in length.

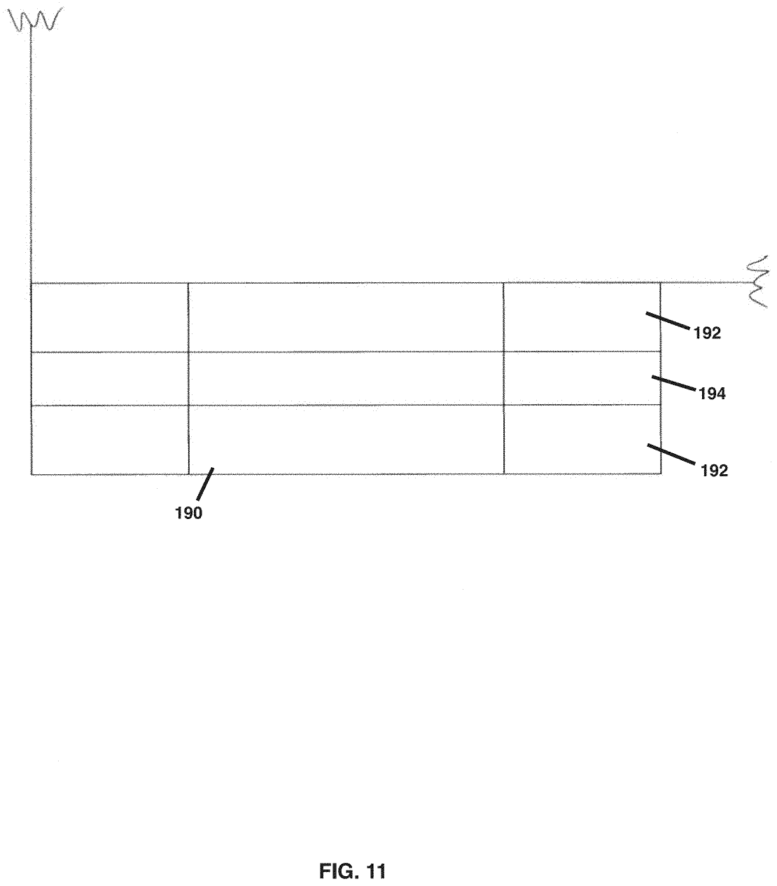

Referring to FIG. 11, each isolation foot 190 may include, for example, layer(s) of elastomeric material 192 and layer(s) of rigid material 194 arranged in a stacked configuration. The elastomeric material serves to dampen and/or dissipate vibrational energy from the booth, whereas the rigid material imparts structural rigidity. A suitable elastomeric material may include, for example, hard rubber. A suitable rigid material may include, for example, porcelain. In the embodiment shown, the isolation feet each include a layer of porcelain sandwiched in between layers of hard rubber.

It is contemplated that the booth may find particular utility within the context of Ayurveda and/or TCM. As those skilled in the art will appreciate, Ayurveda and TCM practitioners believe that the Ayurveda and TCM energy systems are affiliated with, and can therefore be affected by, certain organs within the body (e.g., the endocrine system) (see FIG. 11). Further, it is also believed that all living and nonliving things are in a constate state of vibration, and that everything has its own unique vibrational energy. And by this principal, it may be possible to target specific organs within the body by stimulating the body's auditory and/or visual senses with specific wavelengths of sound and light (i.e., tone and color). Upon positive sensory stimulation of these organs, it may be possible to unblock the blockages in the energy systems of a person and bring those energy systems back into a state of balance.

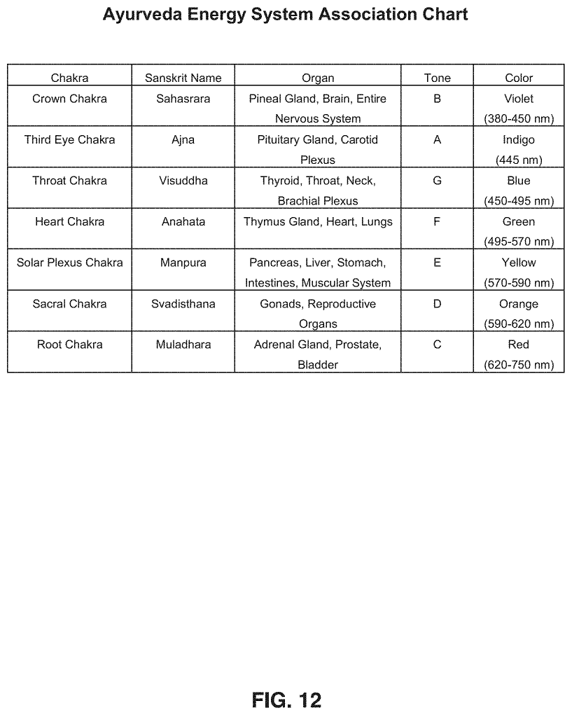

A method of using the booth to stimulate the energy systems of a person according to Ayurvedic principals will now be described. The method may begin by first obtaining relevant personal information from a user. This step may entail, for example, having the user complete a chakra evaluation form and/or obtaining the medical records of the user. The user may then step into the booth and the speakers and light fixtures within the booth can be activated to stimulate the auditory and visual senses of the user. The sounds being played by the speakers may include a range of tones that, at the very least, include the notes of a musical major scale (e.g., C, D, E, F, G, A, and B) tuned to any suitable reference pitch (e.g., 432 hertz, 440 hertz, etc.). These notes may be recorded using any suitable instrument (e.g., quartz singing bowl) and at any suitable recording rate (e.g., 44.1 kilohertz or 48 kilohertz, though 48 kilohertz may be preferred). Further, the light being emitted may be of a color that matches the musical note being played (FIG. 12). In this way, each energy system may be targeted and stimulated. The order in which each energy system is stimulated is not meant to be limiting, but it is contemplated that sequential order starting from the root chakra to the crown chakra may be preferred. The length of time in which each energy system is stimulated is also not meant to be limiting, but it is contemplated that a period of time ranging from 2 minutes to 14 minutes may be preferred. At this point, a skilled Ayurveda practitioner (e.g., a physician) may then conduct an exit interview and make assessment regarding which energy systems require additional stimulation based on whether or not blockages/imbalances still remain.

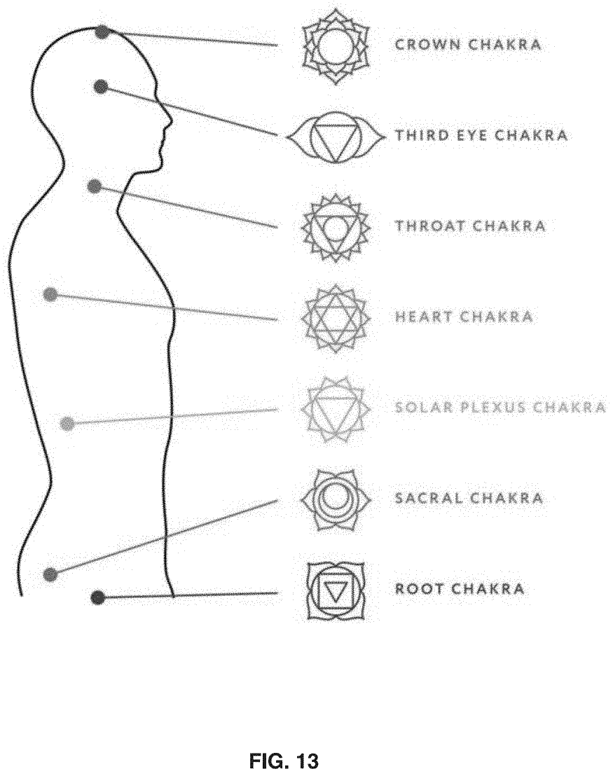

Optionally, the method may also include having the user utilize a specialized seat that features seven bass shakers aligned in correspondence with the seven chakras of Ayurveda (FIG. 13). That is to say, the bass shakers would be arranged proximate (i.e., on or near) the seat (which would necessarily require a back) such that each bass shaker could produce and direct vibrations towards that anatomical part of the user that corresponds with the location of each charka shown in FIG. 13. In this way, the user could target each charka not only with sound and light, but with tactile vibration as well.

Additionally or alternatively to the above, it is also contemplated that the booth can be used to change brain wave frequency modulation. As those skilled in the art will appreciate, brain wave frequencies can generally be categorized as follows: delta (0.1 to 3.5 hertz), theta (4-8 hertz), alpha (8-12 hertz), beta (12-30 hertz), and gamma (above 30 hertz). These brain wave frequencies can be associated with various mental states ranging from deep stillness and meditation (delta and gamma) to active learning and quick response (gamma). Through use of the booth, it may be possible to alter and/or facilitate the changing of brain wave frequencies so as to enable the user to achieve a desired mental state.

Additionally or alternatively to the above, it is also contemplated that the booth can be used to regulate global homeostasis through specific subcutaneous stimulation of the photo endocrine system, stimulate and trigger the autoimmune system favorably, and reduce anxiety and promote feelings of wellbeing with regular use.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It is the intention, therefore, to limit the invention only as indicated by the scope of the claims.

What is claimed is:

1. A sensory stimulation booth comprising:
an enclosure comprising a floor, a ceiling, and a plurality of walls positioned around the periphery of the floor and the ceiling to collectively define an interior space therebetween;
a plurality of speakers within the interior space;
a plurality of light fixtures within the interior space; and
wherein each speaker and each light fixture is oriented towards a focal point within the interior space.

2. The booth of claim 1, wherein the light fixtures comprise RGBW LED lights.

3. The booth of claim 1, wherein the light fixtures comprise lights having a brightness ranging from about 8,000 lux to about 12,000 lux.

4. The booth of claim 1, wherein the speakers comprise speakers having a sensitivity rating of less than 100 decibels.

5. The booth of claim 1, wherein the speakers comprise speakers having a frequency range of about 1 hertz to about 20,000 hertz.

6. The booth of claim 1 further comprising carpeting within the interior space, the carpeting being positioned on at least one of the floor and the ceiling.

7. The booth of claim 1, further comprising a seat within the interior space positioned at the focal point, wherein the seat comprises at least one tactile transducer.

8. The booth of claim 7, wherein the tactile transducer is positioned beneath the seat and is oriented upwards.

9. The booth of claim 7, wherein:
the seat is a chair having a backrest; and
the seat comprises a plurality of tactile transducers aligned in a vertical row along the backrest of the chair.

10. The booth of claim 7 wherein the tactile transducer is configured to produce vibrations having a frequency ranging from about 5 hertz to about 200 hertz.

11. A sensory stimulation booth comprising:
an enclosure comprising a floor, a ceiling, and a plurality of walls positioned around the periphery of the floor and ceiling in a polygonal arrangement, with the floor, ceiling, and walls defining an interior space therebetween;
a plurality of speakers within the interior space;
a plurality of light fixtures within the interior space; and
wherein each speaker and each light fixture is oriented towards a focal point within the interior space.

12. The booth of claim 11, wherein the plurality of speakers is arranged within the interior space such that the plurality of speakers surrounds the focal point of a plurality of different directions.

13. The booth of claim 11, wherein the plurality of light fixtures is arranged within the interior space such that the plurality of light fixtures surrounds the focal point of a plurality of different directions.

14. The booth of claim 11, wherein:
the walls define a number of corners within the interior space; and
a speaker of the plurality of speakers is oriented towards a corner.

15. The booth of claim 11, wherein the ceiling is a vaulted ceiling.

16. The booth of claim 15, wherein the vaulted ceiling comprises a plurality of sloping sides oriented at a slope angle ranging from 3°-20°.

17. The booth of claim 11, further comprising a plurality of isolation feet beneath the floor of the enclosure.

18. The booth of claim 17, wherein the isolation feet comprise a layered configuration comprising at least one layer of rubber and at least one layer of ceramic.

19. A sensory stimulation booth comprising:
an enclosure comprising a floor, a vaulted ceiling, and a plurality of walls positioned around the periphery of the floor and the vaulted ceiling in a hexagonal arrangement, with the floor, vaulted ceiling, and walls defining an interior space therebetween;
a plurality of isolation feet underneath the floor of the enclosure;
a plurality of speakers within the interior space;
a plurality of light fixtures within the interior space; and
wherein each speaker and each light fixture is oriented towards a single point within the interior space.
